(19) United States
(12) Patent Application Publication
(10) Pub. No.: US 2021/0357755 A1
(43) Pub. Date: Nov. 18, 2021

(54) MEDICAL DEVICE ANALYSIS SYSTEM, MEDICAL DEVICE ANALYSIS METHOD, AND LEARNED MODEL

(71) Applicant: OLYMPUS CORPORATION, Tokyo (JP)

(72) Inventor: Takuya HANASHI, Mitaka-shi (JP)

(73) Assignee: OLYMPUS CORPORATION, Tokyo (JP)

(21) Appl. No.: 17/388,337

(22) Filed: Jul. 29, 2021

Related U.S. Application Data
(63) Continuation of application No. PCT/JP2019/003337, filed on Jan. 31, 2019.

(51) Int. Cl. G06N 3/08; G06N 3/04; G06F 16/28; G16H 40/40; G16H 40/67; A61L 2/24; A61L 2/18

(52) U.S. Cl. CPC G06N 3/08; G06N 3/04; G06F 16/284; G16H 40/40; A61L 2202/24; A61L 2/24; A61L 2/18; A61L 2202/14; A61L 2202/17; G16H 40/67

(57) ABSTRACT

A medical device analysis system includes: a medical device; a learning medical device; and a processor comprising hardware. The processor is configured to input multidimensional structural information of the medical device, estimate a residual contamination status after cleaning of the medical device from the input multidimensional structural information of the medical device, based on a learned model that learns about a relationship between the multidimensional structural information of the learning medical device and the residual contamination status after cleaning of the learning medical device, and output the estimated residual contamination status after cleaning of the medical device.

### CROSS REFERENCE TO RELATED APPLICATIONS

The present application is a continuation application based on a PCT Patent Application No. PCT/JP2019/003337, filed on Jan. 31, 2019, the entire content of which is hereby incorporated by reference.

### BACKGROUND

### Technical Field

The present invention relates to an analysis system, an analysis method, and a learned model of a medical device that is reused and requires cleaning.

### Background

For medical devices and plumbing fittings that require cleaning for reuse, it is necessary to predict the cleanability at the time of reuse at the design stage and design a shape that is easy to clean. In predicting detergency, it is necessary to consider various conditions such as the structural information of the system, the usage conditions of the system, and the cleaning conditions.

Japanese Patent (Granted) Publication No. 4835944 (hereinafter referred to as Patent Document 1) describes a parameter value calculation method in pipe cleaning in which cleaning parameters are estimated by fluid simulation. According to the parameter value calculation method in pipe cleaning described in Patent Document 1, the cleaning property can be predicted at the design stage of the pipe structure.

### SUMMARY

A medical device analysis system, includes: a medical device; a learning medical device; and a processor comprising hardware. The processor is configured to input multidimensional structural information of the medical device, estimate a residual contamination status after cleaning of the medical device from the input multidimensional structural information of the medical device, based on a learned model that learns about a relationship between the multidimensional structural information of the learning medical device and the residual contamination status after cleaning of the learning medical device, and output the estimated residual contamination status after cleaning of the medical device.

A medical device analysis method, includes: dividing multidimensional structural information of a medical device into unit regions to generate a plurality of division multidimensional structural information; generating second multidimensional structural information corresponding to each of the plurality of division multidimensional structural information from the multidimensional structural information of the medical device; and estimating a residual contamination status after cleaning of the medical device from the division multidimensional structural information of the medical device and the second multidimensional structural information corresponding to the division multidimensional structural information, based on the learned model learned about a relationship between the multidimensional structural information of the learning medical device and the residual contamination status after cleaning of the learning medical device.

A learned model is learned about a relationship between a multidimensional structural information of a learning medical device and a residual contamination status after cleaning of the learning medical device. The learned model is configured by a convolutional neural network. Division multidimensional structural information generated by dividing the multidimensional structural information of a medical device into unit regions, and a second multidimensional structural information corresponding to division multidimensional structural information generated from the multidimensional structural information of the medical device are input to an input layer of the convolutional neural network. The learned model makes a computer function to output a residual contamination status after cleaning of the medical device from an output layer of the convolutional neural network.

### BRIEF DESCRIPTION OF THE DRAWINGS

FIG. 1 is an overall configuration diagram of an endoscope to be analyzed by the medical device analysis system according to the embodiment.

FIG. 2 is a diagram showing a functional block of the medical device analysis system.

FIG. 3 is a diagram showing a unit region which is a unit for dividing multidimensional structural information.

FIG. 4 is a diagram showing processing of a resampling unit of the medical device analysis system.

FIG. 5 is a constructive conceptual diagram of a learned model of the medical device analysis system.

FIG. 6 is a diagram showing an example of a display screen of a display unit of the medical device analysis system.

FIG. 7 is a diagram showing teacher data and learning results of a learning endoscope.

FIG. 8 is a flowchart showing the operation of the medical device analysis system.

FIG. 9 is an estimation result of the residual contamination status after cleaning of the endoscope by the medical device analysis system.

FIG. 10 is a diagram showing a residual contamination status after cleaning peculiar to bacteria in an endoscope.

### DETAILED DESCRIPTION

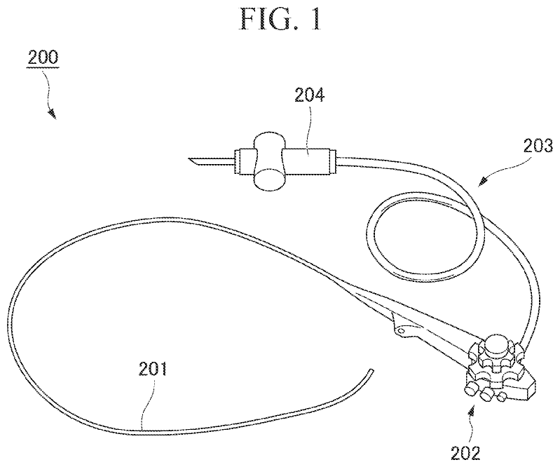

An embodiment of the present invention will be described with reference to FIGS. 1 to 10.

FIG. 1 is an overall configuration diagram of an endoscope (medical device) 200 to be analyzed by the medical device analysis system 100 according to the present embodiment.

### [Endoscope (Medical Device) 200]

As shown in FIG. 1, the endoscope (medical device) 200 includes an insertion portion 201, an operation portion 202, a universal cable 203, and a connector 204.

The insertion portion 201 is an elongated long member that is inserted into the observation target site. At the distal end of the insertion portion 201, an opening (not shown) for air supply/water supply, an illumination optical system provided with a light guide (not shown), and an imaging unit (not shown) provided with an imaging device are provided.

The operation portion 202 has an operation knob and various switches. By operating the operation portion 202, the operator controls air supply/water supply, curvature of the insertion portion 201, and the like.

The universal cable 203 extends from a side part of the operation portion 202. Inside the universal cable 203, an air supply/water supply tube, an illumination optical system provided at the distal end of the insertion portion 201, a cable for telecommunications with the imaging unit, and the like are inserted. A connector 204 is provided at the distal end of the universal cable 203. The universal cable 203 is connected to an external device via the connector 204.

When the endoscope 200 is cleaned after use, the entire endoscope 200 including the universal cable 203 and the connector 204 is subject to cleaning.

### [Medical Device Analysis System 100]

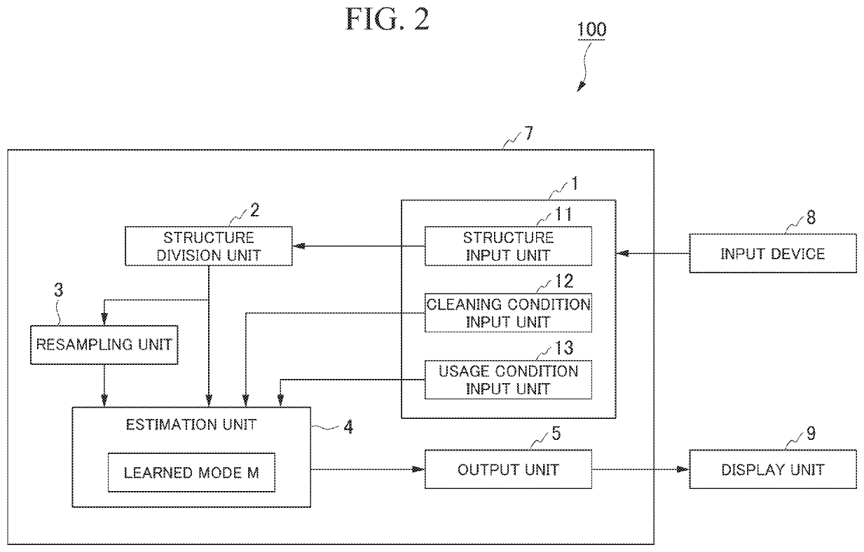

FIG. 2 is a diagram showing a functional block of the medical device analysis system 100 according to the present embodiment.

The medical device analysis system 100 includes a computer 7 capable of executing a program, an input device 8 capable of inputting data, and a display unit 9 such as an LCD monitor.

The computer 7 is a program-executable device including a CPU (Central Processing Unit), a memory, a storage unit, and an input/output control unit. By executing a predetermined program, it functions as a plurality of functional blocks such as an estimation unit 4 described later. The computer 7 may further include a GPU (Graphics Processing Unit), a dedicated arithmetic circuit, and the like in order to process the arithmetic executed by the estimation unit 4 and the like at high speed.

As shown in FIG. 2, the computer 7 includes an input unit 1, a structure division unit 2, a resampling unit (second structure division unit) 3, an estimation unit 4, and an output unit 5. The function of the computer 7 is realized by the computer 7 executing the medical device analysis program provided to the computer 7.

The input unit 1 receives the data input from the input device 8. The input unit 1 includes a structure input unit 11, a cleaning condition input unit 12, and a usage condition input unit 13.

Multidimensional structural information of the endoscope 200 is input to the structure input unit 11. The multidimensional structural information of the endoscope 200 is three-dimensional structural information such as a three-dimensional CAD, and is data capable of specifying a structure in a three-dimensional space. The multidimensional structural information of the endoscope 200 may include the material (rubber, metal, or the like) of each member.

The cleaning conditions for cleaning the endoscope 200 are input to the cleaning condition input unit 12. The cleaning conditions are, for example, the number of brush cleanings, whether or not automatic cleaning is performed, the model and automatic cleaning mode in the case of automatic cleaning, the temperature of the cleaning water, the presence or absence of disinfection, the type of detergent/disinfectant, or the like.

The usage condition input unit 13 inputs the usage conditions of the endoscope 200 when cleaning the endoscope 200. The usage conditions are, for example, the number and time of use of the endoscope 200 since the previous cleaning, the total number of years of use of the endoscope 200, the intended use of the endoscope 200, the number of cleanings, the type of contamination, and the type of contaminants, or the like.

Here, the cleaning conditions and the usage conditions of the endoscope 200 are not essential input data. On the other hand, the multidimensional structural information of the endoscope 200 is indispensable input data.

The structure division unit 2 divides the multidimensional structural information of the endoscope 200 input to the structure input unit 11 into a unit region U to generate a plurality of “division multidimensional structural information D” (division step).

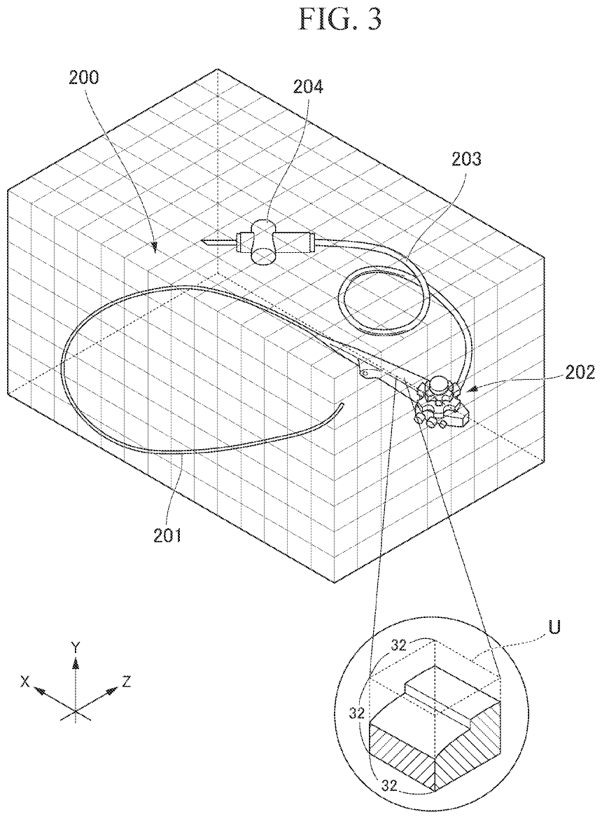

FIG. 3 is a diagram showing a unit region U, which is a unit for dividing multidimensional structural information.

The multidimensional structural information of the endoscope 200 input to the structure input unit 11 is converted into voxels in the three-dimensional space. The voxel in the three-dimensional space is divided for each unit region U to become the division multidimensional structural information D. In the following description, the three axes orthogonal to each other in the three-dimensional space will be referred to as an X-axis, a Y-axis, and a Z-axis.

In the present embodiment, as shown in FIG. 3, the division multidimensional structural information D divided for each unit region U contains the data of 32 voxels in the X-axis direction, 32 voxels in the Y-axis direction, and 32 voxels in the Z-axis direction. In the following description, the area size of such a voxel is expressed as (X, Y, Z)=(32, 32, 32).

The division multidimensional structural information D in the unit region U that does not include the endoscope 200 and the unit region U that does not include the surface of the endoscope 200 even if the endoscope 200 is included does not include the portion to be cleaned. Therefore, it is not subject to estimation of the residual contamination status after cleaning performed in the subsequent treatment. Therefore, the subsequent processing of the division multidimensional structural information D, which is not the target for estimating the residual contamination status after cleaning, is omitted.

The resampling unit (second structure division unit) 3 generates “second multidimensional structural information R” which is multidimensional structural information of the peripheral region including the unit region U from the multidimensional structural information of the endoscope 200. The resampling unit 3 generates two types of second multidimensional structural information R (R1, R2). The second multidimensional structural information R, like the division multidimensional structural information D, includes voxel data in the three-dimensional space.

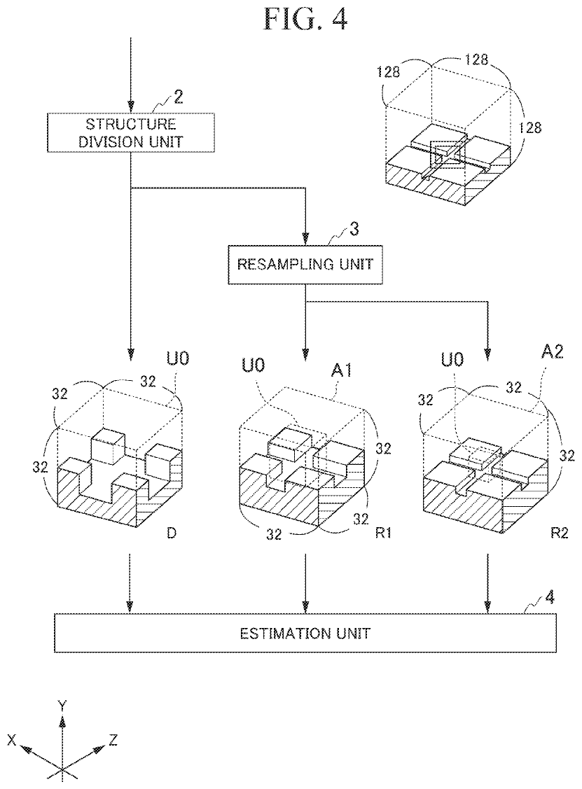

FIG. 4 is a diagram showing the processing of the resampling unit 3.

Multidimensional structural information of the endoscope 200 is input to the structure division unit 2. In FIG. 4, for simplification of the description, only a part of the multidimensional structural information of the endoscope 200 is input, not the whole multidimensional structural information of the endoscope 200. The structure division unit 2 converts the input multidimensional structural information into, for example, a voxel having a region size (X, Y, Z)=(128, 128, 128). The structure division unit 2 divides the converted voxels into the unit region U, and generates a plurality of division multidimensional structural information D. The division multidimensional structural information D includes voxel data having a region size of (X, Y, Z)=(32, 32, 32).

The resampling unit 3 generates two-dimensional structural information R (R1, R2), which is one of a plurality of division multidimensional structural information D output by the structure division unit 2, and corresponds to the division multidimensional structural information D of the unit region U0, which is one of the unit regions U.

The resampling unit 3 generates the second multidimensional structural information R1 of the peripheral region A1 including the unit region U0, which corresponds to the division multidimensional structural information D of the unit region U0. As shown in FIG. 4, the unit region U0 is located at the center in the peripheral region A1.

Further, the resampling unit 3 generates the second multidimensional structural information R2 of the peripheral region A2 including the unit region U0, which corresponds to the division multidimensional structural information D of the unit region U0. As shown in FIG. 4, the unit region U0 is located at the center in the peripheral region A2. Here, the peripheral region A2 is a region including the peripheral region A1. That is, the second multidimensional structural information R2 includes structural information relating to a wider peripheral region as compared with the second multidimensional structural information R1.

The resampling unit 3 also generates the second multidimensional structural information R (R1, R2) corresponding to the division multidimensional structural information D corresponding to each of the unit regions U other than the unit region U0.

In the present embodiment, the second multidimensional structural information R (R1, R2) is information whose resolution has been reduced so that the division multidimensional structural information D and the voxel region size (information amount) are the same. The division multidimensional structural information D and the second multidimensional structural information R (R1, R2) have voxel region sizes of (X, Y, Z)=(32, 32, 32). The division multidimensional structural information D and the second multidimensional structural information R (R1. R2) have the same voxel data area size, and are easy to handle in the subsequent processing of the estimation unit 4.

In the present embodiment, there are two types of second multidimensional structural information R (R1, R2) generated by the resampling unit 3 for one division multidimensional structural information D, but there may be three or more types of information D for one division multidimensional structure. The more types of second multidimensional structural information R to be generated, the higher the accuracy of estimation of the residual contamination status after cleaning by the estimation unit 4.

Based on the “learned model M”, the estimation unit 4 estimates the residual contamination status after cleaning of the endoscope 200 (estimation step) from the multidimensional structural information of the endoscope 200 input to the structure input unit 11, based on the cleaning conditions and the usage conditions.

The learned model M is a convolutional neural network (CNN), which inputs the division multidimensional structural information D and the second multidimensional structural information R generated from the multidimensional structural information of the endoscope 200, the cleaning condition, and the usage condition, and outputs the residual contamination status after cleaning of the endoscope 200. Voxels can be input as input data to the learned model M.

The learned model M is used as a program module of a part of the medical device analysis program executed by the computer 7 of the medical device analysis system 100. The computer 7 may have a dedicated logic circuit or the like for executing the learned model M.

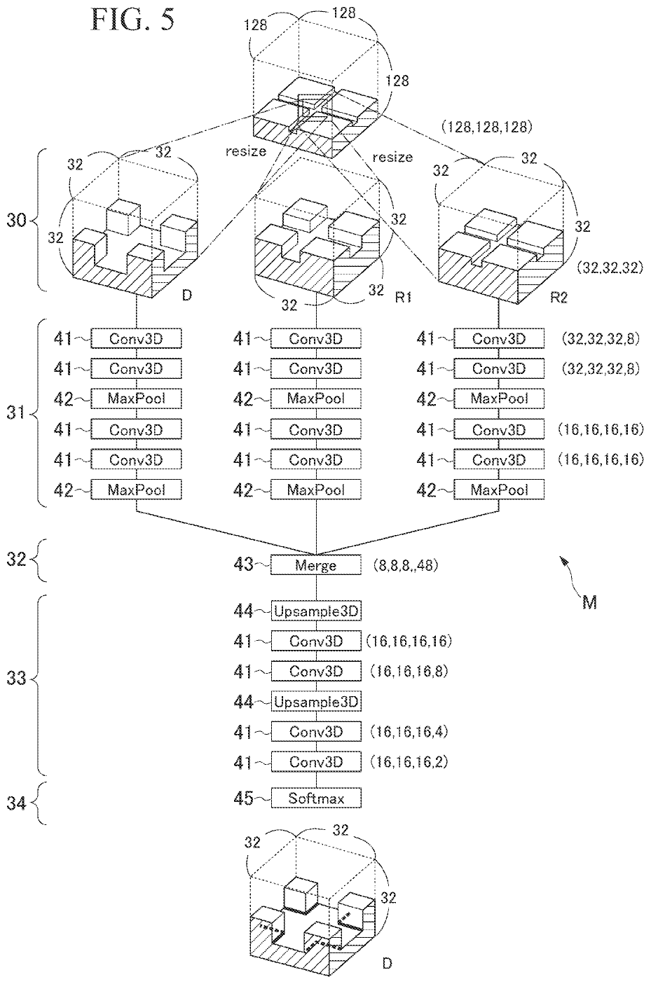

FIG. 5 is a constructive conceptual diagram of the learned model M.

The learned model M includes an input layer 30, a first layer 31, a second layer 32, a third layer 33, and an output layer 34.

The input layer 30 receives the division multidimensional structural information D input from the structure division unit 2 and the second multidimensional structural information R (R1, R2) input from the resampling unit 3. The input layer 30 outputs the division multidimensional structural information DO and the second multidimensional structural information R (R1, R2) to the first layer 31.

The first layer 31 has three networks in which a filter layer (Conv3D) 41 and a pooling layer (MaxPool) 42 are connected in series in parallel. The division multidimensional structural information D and the second multidimensional structural information R (R1, R2) are input to each of the three networks formed in parallel.

The filter layer (Conv3D) 41 performs the image convolution calculation by the learned filter processing obtained by the learning. The activation functions of the nodes of the filter layer are a Step function, a Sigmad function, a ReLU (Rectified Linear Unit) function, a Leaky ReLU function, a Parametric ReLU function, an Exponential linear unit function, a Softsine function, a Tanh function, or the like. In FIG. 5, the argument in parentheses next to the filter layer 41 is a parameter of the filter layer 41. The first argument is the number of voxels in the X-axis direction, the second argument is the number of voxels in the Y-axis direction, the third argument is the number of voxels in the Z-axis direction, and the fourth argument is the number of filters to be applied.

The pooling layer 42 is filtered to reduce the resolution. The pooling layer 42 has a dimension reduction function of reducing the amount of information while retaining the features. The first layer 31 can spatially extract structural information from voxels by alternately repeating the filter layer 41 and the pooling layer 42.

The second layer 32 has a merge layer (Merge) 43 that combines three independent inputs input from the first layer 31. When the merge layer 43 merges the three inputs, correspondence based on the division multidimensional structural information D and the second multidimensional structural information R (R1, R2) input to the first layer 31 being associated with the same unit region U is not essential.

The third layer 33 is a network in which the filter layer (Conv3D) 41 and an upsampling layer (Upsample3D) 44 are connected in series. The upsampling layer 44 performs upsampling on the voxel data.

The output layer 34 has a Softmax function 45. The Softmax function 45 converts the output of the third layer 33 into a residual contamination status after cleaning (binary value) corresponding to the division multidimensional structural information D and outputs the output. The residual contamination status after cleaning (binary value) is a value indicating the presence or absence of residual contamination status after cleaning. The residual contamination status after cleaning (binary value) is output for each voxel.

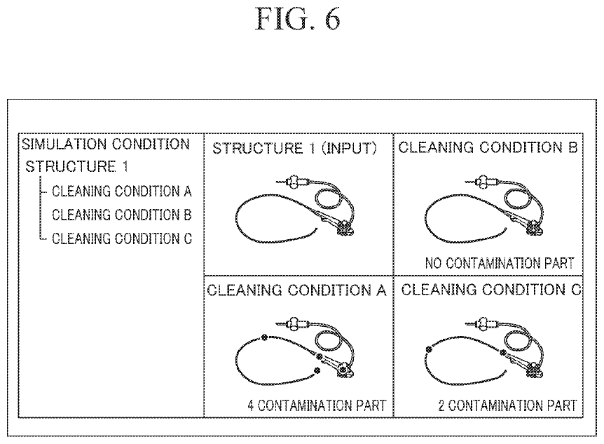

FIG. 6 is a diagram showing an example of a display screen of the display unit 9.

The output unit 5 outputs the residual contamination status after cleaning input from the output layer 34 to the display unit 9. As shown in FIG. 6, the display unit 9 displays the input residual contamination status after cleaning.

### [Generation of Learned Model M]

The learned model M is generated by prior learning based on the teacher data described later. The learned model M may be generated by the computer 7 of the medical device analysis system, or may be performed by using another computer having a higher computing power than the computer 7.

The learned model M is generated by supervised learning by the error back propagation method (backpropagation), which is a well-known technique, and the filter configuration of the filter layer 41 and the weighting coefficient between neurons (nodes) are updated.

In the present embodiment, the teacher data is the residual contamination status after cleaning, which is analyzed by actually cleaning the medical device after use. In the following description, an endoscope that has been used and washed for learning will be referred to as a “learning endoscope (learning medical device)”. Specifically, the combination of the division multidimensional structural information D and the second multidimensional structural information R generated from the multidimensional structural information of the learning endoscope, the cleaning conditions, the usage conditions, and the residual contamination status after cleaning of the learning endoscope is the teacher data. The residual contamination status after cleaning of the learning endoscope is, for example, the place and amount of protein attached.

It is desirable to prepare as diverse teacher data as possible by changing the multidimensional structural information of the learning endoscope and the cleaning conditions and usage conditions. In particular, by preparing teacher data for various cleaning conditions and usage conditions, it is possible to generate a learned model M having high S/N discrimination ability for noise generated under various conditions and capable of estimating the robust residual contamination status after cleaning.

The computer 7 inputs the division multidimensional structural information D and the second multidimensional structural information R generated from the multidimensional structural information of the learning endoscope to the input layer 30, inputs the cleaning conditions and the usage conditions to the second layer 32, and learns the filter configuration of the filter layer and the weighting coefficient between neurons (nodes), so that the mean square error between the residual contamination status after cleaning of the teacher data and the residual contamination status after cleaning output from the output layer 34 is reduced.

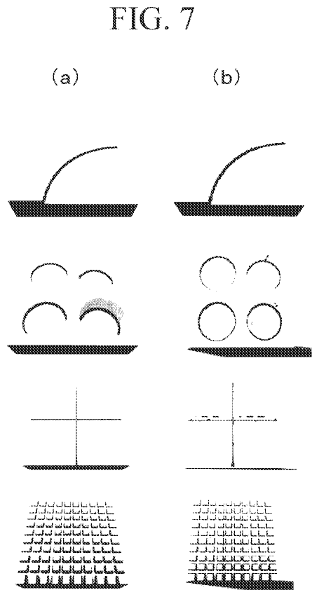

FIG. 7 is a diagram showing teacher data and learning results of the learning endoscope.

(a) of FIG. 7 is a learning endoscope divided into unit regions U. In FIG. 7 (a), a part where the contamination actually remains after cleaning is colored, and is shown as the residual contamination status after cleaning.

(b) of FIG. 7 is a result of estimating the residual contamination status after cleaning of the learning endoscope using the learned model M after learning. The estimation result of the residual contamination status after cleaning shown in FIG. 7 (b) can estimate the residual contamination status after cleaning with a prediction accuracy of 99% or more with respect to the residual contamination status after cleaning shown in FIG. 7 (a). This indicates that the learned model M is a model learned with high accuracy.

### [Operation of Medical Device Analysis System 100]

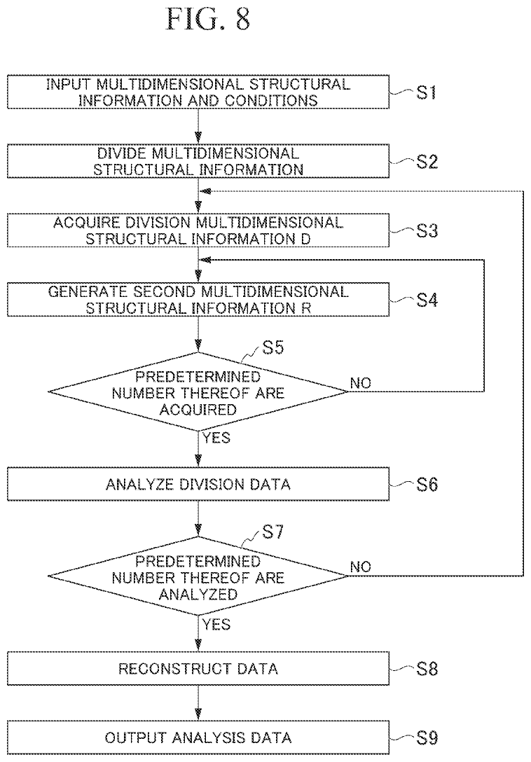

Next, the operation of the medical device analysis system 100 will be described. FIG. 8 is a flowchart showing the operation of the medical device analysis system 100.

In step S1, the computer 7 receives the multidimensional structural information of the endoscope 200 and the input of the cleaning conditions and the usage conditions in the case of cleaning the endoscope 200.

In step S2, the computer 7 converts the multidimensional structural information of the endoscope 200 into voxels in the three-dimensional space. The voxel in the three-dimensional space is divided for each unit region U to become the division multidimensional structural information D.

In step S3, the computer 7 acquires one division multidimensional structural information D from the divided plurality of division multidimensional structural information D.

In step S4, the computer 7 generates the second multidimensional structural information R1 corresponding to the division multidimensional structural information D acquired in step S3. In step S5, the computer 7 determines whether or not a predetermined number of second multidimensional structural information R have been acquired. In the present embodiment, since two types of second multidimensional structural information R are generated, the computer 7 executes step S4 again, and generates the second multidimensional structural information R2 corresponding to the division multidimensional structural information D acquired in step S3.

In step S6, the computer 7 estimates the residual contamination status after cleaning of the endoscope 200 from the division multidimensional structural information D and the second multidimensional structural information R based on the cleaning conditions and the usage conditions based on the learned model M.

In step S7, the computer 7 determines whether or not the residual contamination status after cleaning has been estimated for all of the divided plurality of division multidimensional structural information Ds. When the estimation is not performed for all the division multidimensional structural information Ds, the computer 7 acquires the data of the other division multidimensional structural information Ds in step S3. When all the division multidimensional structural information D are estimated, the computer 7 performs step S8.

In step S8, the computer 7 reconstructs the residual contamination status after cleaning estimated for each of the plurality of division multidimensional structural information Ds, and generates a residual contamination status after cleaning for the entire multidimensional structural information before division.

In step S9, the computer 7 outputs the reconstructed residual contamination status after cleaning to the display unit 9.

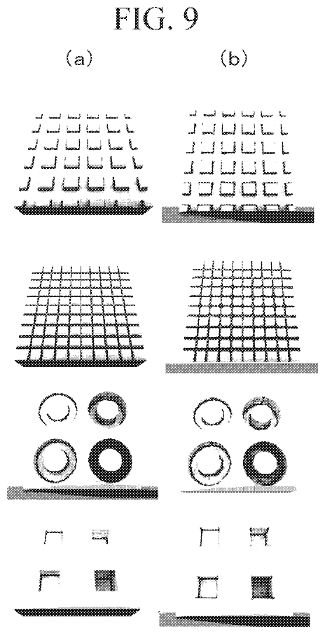

FIG. 9 is an estimation result of the residual contamination status after cleaning of the endoscope 200.

(a) of FIG. 9 is a part of the endoscope 200 divided into the unit region U. Areas where contamination is expected to remain after cleaning are shown colored.

(b) of FIG. 9 is an estimation result of the residual contamination status after cleaning of the endoscope 200. The estimation result of the residual contamination status after cleaning shown in FIG. 9 (b) shows that the residual contamination status after cleaning can be estimated with a prediction accuracy of 99% or more with respect to the residual contamination status after cleaning shown in FIG. 9 (a). The estimation result shown in FIG. 9 (b) shows that the residual contamination status after cleaning of the endoscope 200 can be estimated with high accuracy based on the learned model M learned by using the residual contamination status after cleaning of the learning endoscope as teacher data.

According to the medical device analysis system 100 of the present embodiment, it is possible to predict the detergency with high accuracy even when the multidimensional conception condition, the usage condition, and the like become complicated at the design stage. Since the multidimensional structural information that is input data has a large amount of information, the amount of information processing in learning and estimation increases. Therefore, the multidimensional structural information needs to be divided and processed as the division multidimensional structural information D, but the division multidimensional structural information D lacks information regarding the peripheral area of the divided unit region. According to the medical device analysis system 100 of the present embodiment, by using the second multidimensional structural information R corresponding to the division multidimensional structural information D as auxiliary information for learning and estimation, the detergency of the unit region U can be predicted with high accuracy in consideration of the peripheral region of the unit region U.

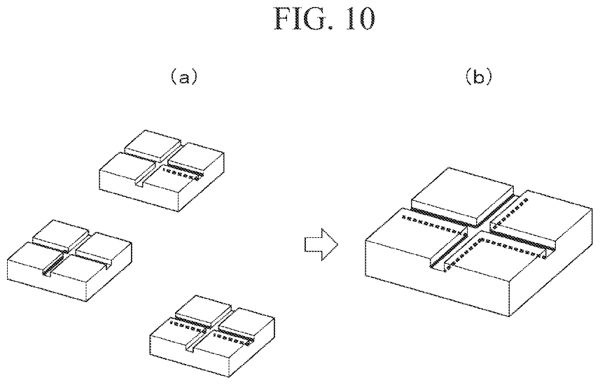

FIG. 10 is a diagram showing the residual contamination status after cleaning peculiar to bacteria of the endoscope 200.

As shown in FIG. 10 (a), the bacteria remaining after cleaning the endoscope 200 are dispersed and adhered in a discrete manner even if the structure and usage conditions are the same, so that it is difficult to estimate the residual contamination status after cleaning, when comparing contamination other than bacteria. However, according to the medical device analysis system 100 of the present embodiment, as shown in FIG. 10 (b), it is possible to generalize and estimate the residual contamination state after cleaning of the bacteria remaining after cleaning the endoscope 200.

Although one embodiment of the present invention has been described in detail with reference to the drawings, the specific configuration is not limited to this embodiment, and design changes and the like within a range not deviating from the gist of the present invention are also included. In addition, the components shown in the above-described embodiment and modification can be appropriately combined and configured.

### Modification Example 1

The function of the medical device analysis system may be realized by recording the medical device analysis program in the above embodiment on a computer-readable recording medium, and having the computer system read and execute the program recorded on the recording medium. The term “computer system” as used herein includes hardware such as an OS and peripheral devices. Further, the “computer-readable recording medium” refers to a portable medium such as a flexible disk, a magneto-optical disk, a ROM, or a CD-ROM, or a storage device such as a hard disk built in a computer system. Further, a “computer-readable recording medium” may also include a program that holds a program for a certain period of time, such as a volatile memory inside a computer system that serves as a server or a client in that case, like a communication line for transmitting a program via a network such as the Internet or a communication line such as a telephone line, and dynamically holds the program for a short period of time.

### Modification 2

For example, in the above embodiment, the learned model M is a convolutional neural network, but the mode of the learned model is not limited to this. The learned model may be a model learned by supervised machine learning such as support vector machine (SVM) linear regression, logistic regression, decision tree, regression tree, and random forest.

What is claimed is:

1. A medical device analysis system, comprising:
a medical device;
a learning medical device; and
a processor comprising hardware,
the processor being configured to input multidimensional structural information of the medical device,
estimate a residual contamination status after cleaning of the medical device from the input multidimensional structural information of the medical device, based on a learned model that learns about a relationship between the multidimensional structural information of the learning medical device and the residual contamination status after cleaning of the learning medical device, and
output the estimated residual contamination status after cleaning of the medical device.

2. The medical device analysis system according to claim 1, wherein
the processor is configured to divide the multidimensional structural information of the medical device into unit regions to generate a plurality of division multidimensional structural information,
generate second multidimensional structural information corresponding to each of the plurality of division multidimensional structural information from the multidimensional structural information of the medical device, and
estimate the residual contamination status after cleaning of the medical device from the division multidimensional structural information and the second multidimensional structural information corresponding to the division multidimensional structural information.

3. The medical device analysis system according to claim 1, wherein
the processor is configured to input a usage condition of the medical device,
the learned model further uses the usage condition of the learning medical device as an input, and
estimates the residual contamination status after cleaning of the medical device from the multidimensional structural information of the medical device based on the usage condition of the medical device.

4. The medical device analysis system according to claim 1, wherein
the processor is configured to input a cleaning condition of the medical device,
the learned model further uses a cleaning condition of the learning medical device as an input, and
estimates the residual contamination status after cleaning of the medical device from the multidimensional structural information of the medical device based on the cleaning condition of the medical device.

5. The medical device analysis system according to claim 1, wherein
the processor is configured to input a usage condition and a cleaning condition of the medical device,
the learned model further uses the usage condition of the learning medical device as an input, and
estimates the residual contamination status after cleaning of the medical device from the multidimensional structural information of the medical device based on the usage condition of the medical device,
the learned model further uses a cleaning condition of the learning medical device as an input, and
estimates the residual contamination status after cleaning of the medical device from the multidimensional structural information of the medical device based on the cleaning condition of the medical device.

6. The medical device analysis system according to claim 2, wherein the second multidimensional structural information is the multidimensional structural information of a peripheral region including the unit region.

7. The medical device analysis system according to claim 2, wherein
the learned model is a model learned about a relationship among the division multidimensional structural information, which is generated from the multidimensional structural information of the learning medical device, the second multidimensional structural information, which is generated from the multidimensional structural information of the learning medical device, and the residual contamination status after cleaning of the learning medical device.

8. The medical device analysis system according to claim 2, wherein
the processor is configured to input a usage condition of the medical device,
the learned model further uses the usage condition of the learning medical device as an input, and
estimates the residual contamination status after cleaning of the medical device from the multidimensional structural information of the medical device based on the usage condition of the medical device.

9. The medical device analysis system according to claim 2, wherein
the processor is configured to input a cleaning condition of the medical device,
the learned model further uses a cleaning condition of the learning medical device as an input, and
estimates the residual contamination status after cleaning of the medical device from the multidimensional structural information of the medical device based on the cleaning condition of the medical device.

10. The medical device analysis system according to claim 6, wherein the second multidimensional structural information is information whose resolution is reduced so that an amount of information is same as that of the division multidimensional structural information.

11. The medical device analysis system according to claim 6, wherein
the learned model is a model learned about a relationship among the division multidimensional structural information, which is generated from the multidimensional structural information of the learning medical device, the second multidimensional structural information, which is generated from the multidimensional structural information of the learning medical device, and the residual contamination status after cleaning of the learning medical device.

12. The medical device analysis system according to claim 10, wherein
the learned model is a model learned about a relationship among the division multidimensional structural information, which is generated from the multidimensional structural information of the learning medical device, the second multidimensional structural information, which is generated from the multidimensional structural information of the learning medical device, and the residual contamination status after cleaning of the learning medical device.

13. A medical device analysis method, comprising:
dividing multidimensional structural information of a medical device into unit regions to generate a plurality of division multidimensional structural information;
generating second multidimensional structural information corresponding to each of the plurality of division multidimensional structural information from the multidimensional structural information of the medical device; and
estimating a residual contamination status after cleaning of the medical device from the division multidimensional structural information of the medical device and the second multidimensional structural information corresponding to the division multidimensional structural information, based on the learned model learned about a relationship between the multidimensional structural information of the learning medical device and the residual contamination status after cleaning of the learning medical device.

14. The medical device analysis method according to claim 13, wherein the second multidimensional structural information is the multidimensional structural information of a peripheral region including the unit region.

15. A learned model that is learned about a relationship between a multidimensional structural information of a learning medical device and a residual contamination status after cleaning of the learning medical device,
wherein the learned model is configured by a convolutional neural network,
division multidimensional structural information generated by dividing the multidimensional structural information of a medical device into unit regions, and second multidimensional structural information corresponding to division multidimensional structural information generated from the multidimensional structural information of the medical device are input to an input layer of the convolutional neural network, and
the learned model makes a computer function to output a residual contamination status after cleaning of the medical device from an output layer of the convolutional neural network.

16. The learned model according to claim 15, wherein the second multidimensional structural information is the multidimensional structural information of a peripheral region including the unit region.

17. The learned model according to claim 15, wherein the convolutional neural network inputs a usage condition of the medical device in addition to the multidimensional structural information.

18. The learned model according to claim 15, wherein the convolutional neural network inputs a cleaning condition of the medical device in addition to the multidimensional structural information.

19. The learned model according to claim 16, wherein the convolutional neural network inputs a usage condition of the medical device in addition to the multidimensional structural information.

20. The learned model according to claim 16, wherein the convolutional neural network inputs a cleaning condition of the medical device in addition to the multidimensional structural information.
